(19) United States
(12) Patent Application Publication
(10) Pub. No.: US 2023/0417721 A1
(43) Pub. Date: Dec. 28, 2023

(54) STEP BY STEP SENSORY RECIPE APPLICATION

(71) Applicant: INTERNATIONAL BUSINESS MACHINES CORPORATION, ARMONK, NY (US)

(72) Inventors: Juel Daniel Raju, Garland, TX (US); Christopher Bryan Barnwell, Grapevine, TX (US); Romelia H. Flores, Keller, TX (US); Susan Jachin Christian, Grapevine, TX (US)

(21) Appl. No.: 17/808,113

(22) Filed: Jun. 22, 2022

(51) Int. Cl. G01N 33/02; G01N 33/00; G06F 3/01

(52) U.S. Cl. CPC G01N 33/02; G06F 3/011; G01N 33/0001

(57) ABSTRACT

According to one embodiment, a method, computer system, and computer program product for aiding a user in the preparation and completion of a dish within a social sensory recipe program are provided. The present invention may include capturing one or more characteristics comprising an aroma of a dish associated with a recipe using one or more sensors, wherein the one or more sensors comprise an olfactory sensor; identifying, at two or more steps of the recipe comprising a recipe profile, one or more aroma profiles of the dish based on the captured one or more characteristics comprising the aroma; comparing the one or more aroma profiles of the dish to one or more aroma profiles comprising the recipe profile; and responsive to the comparing, providing feedback of the dish to a user.

### BACKGROUND

The present invention relates, generally, to the field of computing, and more particularly to digital gastronomy.

Gastronomy refers to the study of food and culture and includes the practices of cooking techniques, nutritional facts, food science, and food tastes and smells. The prominent role of technology in society allows for traditional cooking to be infused with computational abilities to create digital gastronomy. With the implementation of digital gastronomy, cooks of all skill levels can enhance their cooking with new digital capabilities. Digital gastronomy has the potential to benefit numerous aspects of food preparation and food outcomes in the kitchen.

### SUMMARY

According to one embodiment, a method, computer system, and computer program product for aiding a user in the preparation and completion of a dish within a social sensory recipe program is provided. The present invention may include capturing one or more characteristics comprising an aroma of a dish associated with a recipe using one or more sensors, wherein the one or more sensors comprise an olfactory sensor; identifying, at two or more steps of the recipe comprising a recipe profile, one or more aroma profiles of the dish based on the captured one or more characteristics comprising the aroma; comparing the one or more aroma profiles of the dish to one or more aroma profiles comprising the recipe profile; and responsive to the comparing, providing feedback of the dish to a user.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates an exemplary networked computer environment according to at least one embodiment;

FIG. 2 is an operational flowchart illustrating a social sensory recipe process according to at least one embodiment;

FIG. 3 is a system diagram illustrating an exemplary program environment of an implementation of a social sensory recipe process depicted according to at least one embodiment;

FIG. 4 is an operational flowchart illustrating a recipe profile creation process according to at least one embodiment;

FIG. 5 depicts a visual representation of a graphical user interface of the system according to at least one embodiment;

FIG. 6 depicts a visual representation of a graphical user interface of the system according to at least one embodiment;

FIG. 7 is a block diagram of internal and external components of computers and servers depicted in FIG. 1 according to at least one embodiment;

FIG. 8 depicts a cloud computing environment according to an embodiment of the present invention; and

FIG. 9 depicts abstraction model layers according to an embodiment of the present invention.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. In the description, details of well-known features and techniques may be omitted to avoid unnecessarily obscuring the presented embodiments.

Embodiments of the present invention relate to the field of computing, and more particularly to the field of digital gastronomy. The following described exemplary embodiments provide a system, method, and program product to, among other things, utilize digital capabilities to assist food preparation and recipe completion. Therefore, the present embodiment has the capacity to improve the technical field of digital gastronomy by providing a program that can aid in the preparation and completion of a recipe using one or more sensors to capture aroma profiles of ingredients and/or dishes, compare the aroma profiles of dishes and provide step by step feedback to users.

As previously described, gastronomy refers to the study of food and culture and includes the practices of cooking techniques, nutritional facts, food science, and food tastes and smells. The prominent role of technology in society allows for traditional cooking to be infused with computational abilities to create digital gastronomy. With the implementation of digital gastronomy, cooks of all skill levels can enhance their cooking with new digital capabilities, allowing for the comparison of dish's aroma profiles, step by step feedback, and the incorporation of diner preferences. Digital gastronomy has the potential to benefit numerous aspects of food preparation and food outcomes in the kitchen.

One such context where digital gastronomy may be poised to make an impact is in user assistance; in situations where a user performs a series of steps to complete a dish, digital gastronomy stands to provide a beneficial impact by comparing the aroma profiles of dishes and providing step by step feedback to users. Currently, several deficiencies exist in food preparation. One of these deficiencies is the difficulty of getting the expected flavor in a dish, especially when a step in a recipe is omitted or performed incorrectly. Additionally, it is difficult to make a dish the correct way every time because of factors such as using too much or too little of an ingredient or modifying a recipe based on preferences and not adjusting the rest of a recipe appropriately. Ultimately, cooks may not have a way to ensure that they will be achieving the expected flavor profile of a dish, especially on a consistent basis. As such, it may be advantageous to, among other things, implement a system that addresses the above deficiencies, including using a variety of sensors for aroma profile comparisons at each step of a recipe profile, modifying a recipe profile based on diner preferences, and providing feedback during the preparation of a recipe profile.

According to one embodiment, the invention is a system, method, and/or computer program product for utilizing data from one or more sensors and feedback to assist a user in the preparation and completion of a dish. The program assists the user through capturing aromas of an ingredient/dish using a variety of sensors, comparing aroma profiles of a dish to aroma profiles of a recipe profile at each step of the recipe profile, receiving feedback during the preparation of a recipe profile, and suggesting modifications to the recipe profile based on the feedback.

In some embodiments of the invention, the social sensory recipe program, herein referred to as “the program”, can receive a recipe profile request from a user. A user can be any person preparing a dish corresponding to a recipe profile. A recipe profile request can comprise receiving a request from a user to make a recipe profile and/or a dish associated with the recipe profile. Receiving a recipe profile request from a user may involve the program receiving a recipe profile request from the online platform in response to a user selecting a recipe profile from the online platform or a user selecting a saved or previously viewed recipe profile from the user's own profile. A recipe profile can represent a recipe for making a dish. A dish may herein refer to the food product being prepared by a user in accordance with the recipe profile. In addition, a recipe profile may include the aroma profile of the dish at each step and may also include aroma values associated with each ingredient for a recipe step. The aroma profile of a dish can comprise the aromas of a dish and one or more aroma values associated with ingredients comprising the dish representing the contribution of the ingredient to the aroma of the dish. An ingredient's aroma profile may comprise the different aromas of an ingredient at multiple stages of freshness. A recipe profile may also include other information pertaining to the dish, such as portion sizes of ingredients comprising the dish, weights of ingredients comprising the dish, cooking temperatures associated with steps of the recipe, suggestions for carrying out steps of the recipe profile, reviews of the dish by users, diners, and other individuals, etc.

In some embodiments of the invention, the program may provide initial feedback to the user based on diner preferences and the recipe profile requested. Initial feedback may be feedback communicated to a user before the user begins executing the recipe profile. The initial feedback may be created based on diner preferences and the applicability of those diner preferences to the requested recipe profile. The initial feedback may comprise suggestions regarding modifications to the recipe profile, such as the addition/subtraction of ingredients, changes in subsequent recipe profile steps, etc. Suggestions created during the initial feedback step may guide a user to the completion of a dish by modifying the recipe profile with respect to the diner preferences. In some embodiments, for example, where a user is not a diner, suggestions created during the initial feedback step may guide a user to the completion of a dish by modifying the recipe profile based on both diner preferences and user preferences. The program may receive diner preferences from the database and/or the online platform. A diner may be a person for whom a user is preparing a dish. Diner preferences can be information the program examines when a recipe profile request is received in order to determine if modifications to the recipe profile will be needed. Diner preferences may be general, such as a diner's ingredient likes/dislikes, including a diner's desire for a greater quantity of a certain ingredient in all dishes prepared for him/her, or a diner's medical conditions that are triggered by certain ingredients, such as allergies. For example, a diner's profile may include the fact that the diner is allergic to basil, and as such, list that basil needs to be excluded from all recipe profiles prepared for them. Additionally, diner preferences may be specific, such as diner feedback. Diner feedback may be comprised of suggested modifications to a recipe profile based on the diner previously consuming the dish corresponding to the recipe profile and communicating dish modifications to the program. Diner feedback may be added to the diner's profile. For example, a diner who had previously consumed a steak dish may communicate diner feedback to the program stating that the diner would prefer the steak to be cooked well-done in the future, which may be uploaded to the diner's profile. The next time the program receives a recipe profile request from a user who is preparing the dish for the diner, the program may examine the database and/or online platform, determine that the recipe profile needs to be modified based on checking a list of uploaded modifications comprised within the diner's profile, and rework the recipe profile to alert a user that the steak should be cooked well-done. Some diner preferences, such as medical conditions, may require a user to respond to an additional pop-up or alert, while other diner preferences, such as ingredient likes/dislikes and diner feedback, may be merely suggested recipe profile modifications that a user could accept or reject.

In some embodiments, a diner may be a user and/or a user may be a diner. In some embodiments, a diner is not a user and/or a user is not a diner. In some embodiments, for example, where a user is not a diner, the program may receive user preferences in addition to diner preferences with respect to the recipe profile. User preferences may record certain ingredients which risk causing an illness/condition to the user because of skin contact and/or smell, such as contact allergies and hyperosmia respectively. Determining the applicability of diner preferences to the requested recipe profile may require the program to assess the requested recipe profile's ingredients and use the assessment in the determination of whether any of the ingredients are encompassed within the diner's preferences, to determine if the requested recipe profile needs to be adjusted to accommodate to the diner's preferences before a user begins executing the recipe profile. After determining that the diner preferences apply to the requested recipe profile, the program may determine what aspects of the recipe profile require modification. The program can automatically recognize whether adding/subtracting certain ingredients or new additional steps will change how a different step will be performed.

In some embodiments of the invention, the program may receive captured physical and/or chemical characteristics of a dish from sensors. The captured characteristics may comprise physical and chemical characteristics of the ingredients in the dish, for example, the smell of an ingredient or the weight of an ingredient, and the dish itself, as combined ingredients may have properties that no individual ingredient possesses, for example, bread, and may also include optical information, temperature, etc. The capturing process may be performed once per step, multiple times per step, multiple times during the preparation of the dish, and/or continuously in real-time or near-real-time by a sensor. The capturing process may also be triggered at the completion of a step by a user answering a prompt. The sensor may be any device or combination of devices enabled to capture physical and/or chemical characteristics of ingredients. The sensor may be any device or combination of devices enabled to communicate the captured physical and/or chemical characteristics of ingredients to the program, for instance via a network, either directly or via some number of proxies or intermediary programs or devices. The sensor may be, for example, an olfactory sensor, digital scale, digital thermometer, microphone, video camera, etc. The sensor may, for example, capture the weight of an ingredient by comprising a scale and transferring the measured weight to the program. Additionally, for example in embodiments where the sensor comprises an olfactory sensor, the sensor may capture the aroma of an ingredient, or dish, and transfer the aroma value, or values, to the program. Olfactory sensors can detect, identify, and quantitatively measure chemicals. Specifically, olfactory sensors can identify the composition of chemical substances in aromas and may provide an objective index. Olfactory sensors may comprise receptor proteins that respond to specific aromas. The olfactory sensor capturing process can begin when an aroma molecule binds to a sensor. Upon binding to an aroma molecule, the sensor can experience a change in electrical properties which convert to a plurality of electric signals by the sensor. A specific response can be recorded by the electronic interface transforming the signal into a digital value and may provide the digital value to a computer system. The computer system may analyze information and provide a pattern recognition output describing the aroma. Olfactory sensors may be coated using different substances to provide unique response characteristics thereto for differing aromas. Additionally, olfactory sensors may be immersed to detect aromas in liquid. Olfactory sensors may be used inside a cooking appliance. Olfactory sensors can allow for an individual to obtain aroma data in both qualitative and quantitative forms.

In some embodiments of the invention, the program may receive captured characteristics using live video and/or augmented reality devices. For example, optical sensors may be used to help capture characteristics such as portion sizes. An optical sensor may capture images from a video feed and the program may use machine vision techniques to identify the ingredients within the captured video footage. The use of live video may allow for a user to be viewed by others while preparing a recipe profile either in real-time or near-real-time. The aroma profile of the dish being prepared may be displayed on the video display with text identifying the percentage of each ingredient in a dish. Through the social platform, others may view the user preparing a recipe profile.

In some embodiments of the invention, the program may visualize different aromas emanating from a user's dish at each step of the recipe profile and display them, using artificial intelligence systems such as IBM Crypto Anchor Verify™ (Crypto Anchor Verifier™ and all Crypto Anchor Verified™_based trademarks and logos are trademarks or registered trademarks of International Business Machines Corporation, and/or its affiliates). The program may visualize different aromas by using a sensor to scan an ingredient and/or dish to capture the components and sub-components. Aroma visualization may allow a user to visually identify the make-up of an ingredient and/or a dish by graphically representing the ingredients/culinary processes that are components of the aroma.

In some embodiments of the invention, the program may determine the freshness of an ingredient using artificial intelligence systems such as IBM Crypto Anchor Verify™ (Crypto Anchor Verifier™ and all Crypto Anchor Verified™_based trademarks and logos are trademarks or registered trademarks of International Business Machines Corporation, and/or its affiliates). Freshness detection may allow a user to check the aroma of each ingredient in a recipe profile to determine how fresh an ingredient is before a user begins executing a recipe profile. Freshness detection can be determined by using a sensor to capture the aroma of an ingredient and then comparing the captured aroma to the ingredient's aroma profile comprised within the aroma composition database. An ingredient's aroma profile may comprise the different aromas of an ingredient at multiple stages of freshness. Freshness detection may be used to help a user better understand the qualify/freshness of an ingredient. The aroma database may contain information pertaining to how aromas of different ingredients change based on the freshness of the ingredient.

In some embodiments of the invention, the program may compare the captured aromas of a dish with the aroma profile comprised within the requested recipe profile. The comparison may involve a quantitative analysis comparing the aroma profile of the user's dish to the aroma profile comprised within the recipe profile. The program can perform the aroma profile comparison by matching the aroma profile of the user's dish to an aroma profile comprised within the recipe profile to determine the current step the user is on in the recipe profile. Once the program matches the aroma profile of the dish with the aroma profile corresponding to the current step comprised within the recipe profile, the program can compare the aroma values of each ingredient in the aroma profile of the user's dish with the aroma values of each ingredient in the aroma profile at the matching step within the recipe profile. Additionally, a user interface may display the current aroma value of an ingredient in a user's dish as a percentage in one column and display the aroma value of the ingredient in a recipe profile's dish as another percentage in another column.

In some embodiments of the invention, the program may identify the individual ingredients of a dish and/or the contribution of each ingredient to the aroma profile of the dish. The program can identify aromas by comparing the captured characteristics against the aroma values comprised within the aroma composition database to identify aromas in the user's dish and/or individual components of the user's dish. The comparison may involve the program matching the individual aroma values of the dish measured at the current step by the olfactory sensor to the aroma values of ingredients comprised within the aroma composition database.

In some embodiments of the invention, the program may provide feedback to a user based on the comparison between the aroma profile of the user's dish and the aroma profile comprised within the recipe profile. The feedback may be generated based on the differences in the aroma profiles between the user's dish and the recipe profile. The program can automatically determine suggested modifications for a user to make to the recipe profile to result in the aroma profile of the user's dish matching the aroma profile comprised within the recipe profile. The program may present the suggested modifications to the user as feedback. The program may provide feedback by displaying alerts/flags to the user. Alerts and flags may be pop-up prompts on the display with text identifying the feedback. For example, a feedback request may be displayed to the user as they are executing a recipe profile step. In some embodiments, the program may provide feedback by means of emitting sounds/speech through the user's mobile or computing device speakers, or by means of initiating vibration of the user's mobile or computer device. The feedback may comprise suggestions regarding modifications to the recipe profile, such as addition/subtraction of ingredients, changes in subsequent recipe profile steps, etc.

In some embodiments of the invention, the program may capture the aroma profile of a user's finished dish, which may involve capturing the aroma profile of the finished dish at the last step of the recipe profile and storing it as part of the aroma profile comprised within the recipe profile.

In some embodiments of the invention, the program may share the aroma profile of the finished dish with the online platform. The online platform may comprise a social platform, the aroma composition database, and/or a social media platform. The online platform may store recipe profiles, store user and/or diner profiles, and/or receive recipe profile requests. The social platform may comprise a social network and may allow for feedback and communication from users and experts, which may be used by users to help improve their dishes. The social media platform may allow for integration of outside platforms so that text and/or pictures may be displayed from outside platforms within the social platform. Outside platforms, for example, may be social networks that are not located within the online platform. The program may share the aroma profile of a finished dish by uploading the aroma profile of the finished dish to the database. By uploading the aroma profile, the aroma profile may be stored and shared on the online platform. The aroma composition database comprises aroma profiles of one or more recipe profiles, which are uploaded by experts. In some embodiments, the aroma profile may be uploaded directly to the social platform and downloaded to the database from the social platform. In some embodiments, the aroma profile may be uploaded to both the aroma compositions database and the social platform. Through the social platform, the uploaded aroma profiles may be both viewed and shared, allowing for comparisons between users' dishes.

In some embodiments of the invention, the program may receive initial information pertaining to a dish from an expert. An expert can be any person who is uploading a recipe profile to the online platform. Initial information can be a set of data corresponding to the preparation of a dish entered by the expert prior to preparing the dish. Initial information may be part of a recipe profile comprising information for the making of a dish, and may correspond to the steps of the recipe profile, the kitchen appliances/tools needed for the recipe profile, what ingredients and how much of each ingredient are needed for the recipe profile, cooking temperatures and times, feedback to help users complete certain steps of the recipe profile, etc.

In some embodiments of the invention, the program may provide feedback to a user. Feedback may be in the form of tips and/or techniques associated with each step of the recipe profile and may be in the form of a video or augmented reality. The forms of feedback offered may include advice for completing a step in the recipe profile in a more efficient manner or ways of using kitchenware. Feedback may be directed toward improving the dining experience of a diner. Feedback is enabled by the program mapping the recipe profile captured characteristics as outlined in recipe profile steps and in the preparation process of a recipe profile's dish. For example, if a user is preparing a Garlic Herb Baked Chicken dish from a recipe profile, the aroma capture of the step where the user rubs herb seasoning over the chicken and the aroma capture of the step after the chicken has been baked, could be shared with another user who would then understand that the aroma capture at the latter step is affected based on the aroma capture in the former step. In addition, the program may determine that the latter step is affected by an ingredient used in the previous step and provide feedback to help the user as they cook using artificial intelligence systems. Feedback may help to improve both the understanding and cooking of the user. The user now may understand that the aroma profile at the herb-rubbing step was indicative of a particularly over-seasoned dish. The guided approach may provide a conducive method of cooking improvement.

In some embodiments of the invention, the program may utilize augmented reality capabilities to enable a user to interact with an expert or another user in real-time or near-real-time using augmented reality capabilities. Augmented reality is a modern computing technology that uses software to generate images, sounds, haptic feedback, and other sensations to augment a real-world environment. The program may use augmented reality to, for example, visually represent the elements of a recipe profile step. The visual representation of a recipe profile may allow a user to get real-time or near-real-time feedback from another user or expert. The user preparing the dish may receive this feedback via text displayed on the augmented reality device in communication with the program. For example, if an expert viewing a user preparing a recipe profile notices the user incorrectly performing a step, the expert may communicate real-time or near-real-time feedback to the user. Augmented reality may be enabled by hardware in use with the program, such as a headset with a sensor incorporated in it or a mobile device with a sensor incorporated in it.

In some embodiments of the invention, a user may be able to send their question to an expert and the expert may be able to directly respond to the user. A feedback request may be requested to submit feedback that is responsive to a user question. For example, if the feedback the user received when they previously served the dish in a recipe profile was, “Would have liked to have had more oregano used in this recipe”, then the program could send a feedback request to the expert to ask, if they had to add more oregano, which step or steps in the recipe profile they would add the oregano to. Additionally, the feedback request may be displayed to the user as they are executing the steps of the recipe profile while the program detects for oregano in the current step and provides feedback to the user to augment the amount of oregano called for in the recipe profile.

In some embodiments of the invention, the program may include real-time or near-real-time cooking information from other users or from suggestions derived from comparing previous olfactory, weight, and portion captures at each step of a recipe profile to the user's dish at the current recipe profile step.

In some embodiments of the invention, the program may allow a user to compare their dish's aroma profile to that of another user or expert, such as their mother or their friend, through the use of a video feature. Through the social platform, others may view the user preparing a recipe profile. The video feature may be live or prerecorded. The use of live video may allow for a user to watch another user or expert create a dish in real-time or near-real-time. A prerecorded video may allow for a user to watch how another user or expert previously prepared a recipe profile. The aroma profile of the dish being prepared may be displayed on the video display with text identifying the percentage of each ingredient in a dish. In some embodiments of the invention, the program may receive captured characteristics using live video. For example, optical sensors may be used to help capture characteristics such as portion sizes. An optical sensor may capture images from a video feed and the program may use machine vision techniques to identify the ingredients within the captured video footage.

In some embodiments of the invention, the program may enable a user to consider the feedback of an individual who has previously consumed the dish prepared from the recipe profile as the user is preparing the recipe profile and provide recommendations to the user based on altering a recipe profile. The feedback may comprise text and/or a rating based on, for example, a 5-star rating system.

In some embodiments of the invention, the program may serve as a standalone application and may be platform agnostic. The program may also be a portable API framework or service if another application integrates the aroma profile capturing and comparison capabilities into their existing application.

In some embodiments of the invention, the program may allow a user to view an interactive user interface. The interactive user interface may allow a user to view a step-by-step process of an expert's dish in a recipe profile showing details about the recipe profile and details about the specific ingredients involved in each step of the recipe profile. The interactive user interface may also allow a user to view more in-depth information about a recipe profile such as generic and specific expert insights.

In some embodiments of the invention, the program may be used in a teaching context. Using the program in a teaching context may comprise a teacher, who is treated as an expert, and one or more students, who are users, who report to the teacher. A teacher may use the program to assess a user. A teacher may assess a user by receiving the aroma profiles of a student's dish and comparing those aroma profiles with the aroma profiles comprised within the recipe profile being taught. Additionally, live video and aroma visualization features may be used in a teaching context. The use of live video may allow a student to watch the teacher create a dish in real-time or near-real-time, or may allow the teacher to watch a student create a dish in real-time or near-real-time. The use of aroma visualization may allow teachers and students to visualize the differences in aromas between the student's dish and the dish corresponding to the recipe profile. Using the program in a teaching context may allow a dish to be prepared in compliance with the recipe profile of the dish being taught in a cooking class and/or guidelines of the cooking class. Guidelines may be created by the teacher regarding food preparation, such as advice on creating a dish, and may be included as feedback in the recipe profile. Feedback generated may be used as a teaching aid, such as for guiding a user to correctly prepare the dish of a recipe profile.

In some embodiments of the invention, the program may be used in an educational context. Using the program in an educational context may comprise a teacher, who is treated as an expert, and one or more students, who are users, who report to the teacher. A teacher may use the program to assess a student. A teacher may assess a student by receiving the aroma profiles of a student's dish and comparing those aroma profiles with the aroma profiles comprised within the recipe profile being taught. Additionally, video and aroma visualization features may be used in an educational context. The use of live video may allow a student to watch the teacher create a dish in real-time or near-real-time, or may allow the teacher to watch a student create a dish in real-time or near-real-time. A prerecorded video may allow a student to watch how another expert previously prepared a recipe profile or watch an expert demonstrate proper cooking techniques. The use of aroma visualization may allow teachers and students to visualize the differences in aromas between the student's dish and the dish corresponding to the recipe profile. Using the program in an educational context may allow a student to learn and practice proper cooking techniques, both in-person and online, and ultimately help a student in preparing consistent dishes.

In some embodiments of the invention, the program may be used in a food service context for enforcing conformity of dishes. Using the program in a food service context may comprise a restaurant having each dish on its menu uploaded as a recipe profile on the program. The restaurant may set up and modify recipe profiles and implement the restaurant guidelines into the program. Guidelines may be created by the restaurant manager regarding safety procedures, tips presented to a user at certain steps, and may be included as feedback in the recipe profile. Using the program in a food service context may allow for dishes to be prepared in compliance with the restaurant recipe profiles and/or restaurant guidelines. Feedback generated by the program may ensure users working at a restaurant are compliant in preparing a dish at all steps of a recipe profile. In the food service context, a user may or may not consider diner feedback. Diner feedback may be divided into different classes of diners, such as diners with food allergies and diners who are gluten-free, etc. Additionally, using the program in a food service context may allow a restaurant with multiple locations, such as chain restaurants or a small business owner having several restaurants, to provide a consistent dish to a diner regardless of which location the diner visits.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method, and program product for utilizing data from one or more sensors, preferences and feedback to assist a user in the preparation and completion of a dish. The program assists the user through capturing aromas of an ingredient/dish using a variety of sensors, comparing aroma profiles of a dish to aroma profiles of a recipe profile at each step of the recipe profile, receiving feedback during the preparation of a recipe profile, and suggesting modifications to the recipe profile based on the feedback.

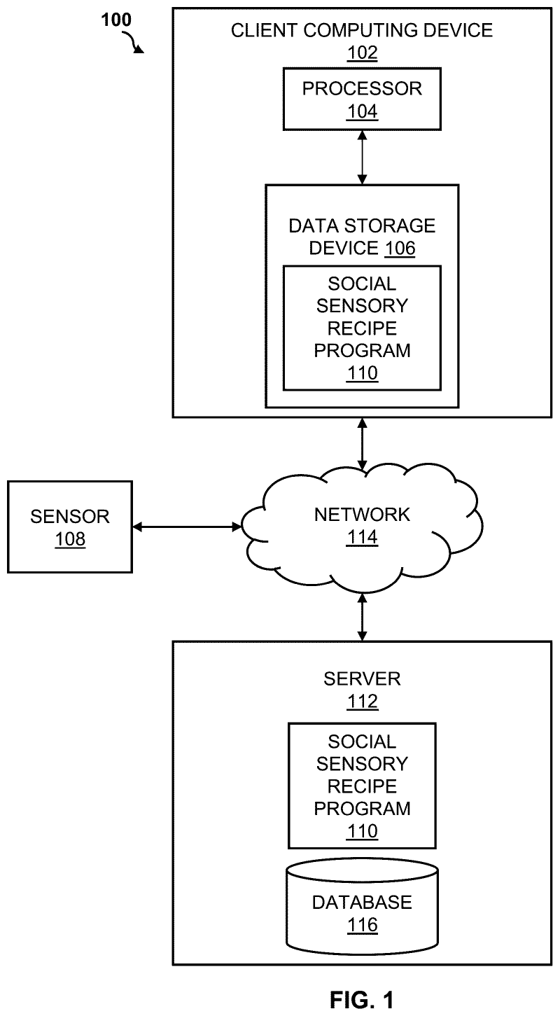

Referring to FIG. 1, an exemplary networked computer environment 100 is depicted, according to at least one embodiment. The networked computer environment 100 may include client computing device 102 and a server 112 interconnected via a communication network 114. According to at least one implementation, the networked computer environment 100 may include a plurality of client computing devices 102, sensors 108 and servers 112, of which only one of each is shown for illustrative brevity.

The communication network 114 may include various types of communication networks, such as a wide area network (WAN), local area network (LAN), a telecommunication network, a wireless network, a public switched network and/or a satellite network. The communication network 114 may include connections, such as wire, wireless communication links, or fiber optic cables. It may be appreciated that FIG. 1 provides only an illustration of one implementation and does not imply any limitations with regard to the environments in which different embodiments may be implemented. Many modifications to the depicted environments may be made based on design and implementation requirements.

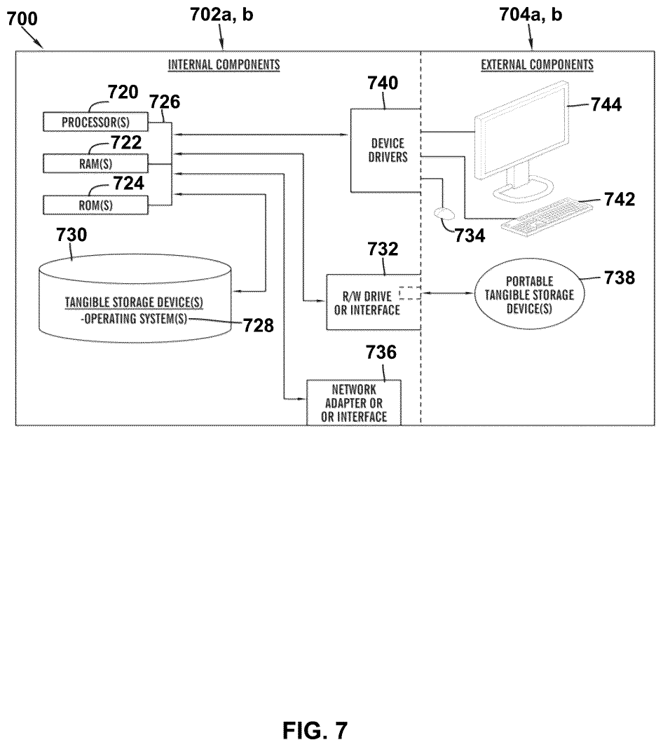

Client computing device 102 may include a processor 104 and a data storage device 106 that is enabled to host and run a social sensory recipe program 110 and communicate with one or more sensors 108 and the server 112 via the communication network 114, in accordance with one embodiment of the invention. Client computing device 102 may be, for example, a mobile device, a telephone, a personal digital assistant, a netbook, a laptop computer, a tablet computer, a desktop computer, or any type of computing device capable of running a program and accessing a network. As will be discussed with reference to FIG. 7, the client computing device 102 may include internal components 702a and external components 704a, respectively.

The server computer 112 may be a laptop computer, netbook computer, personal computer (PC), a desktop computer, or any programmable electronic device or any network of programmable electronic devices capable of hosting and running the program 110 and a database 116 and communicating with the client computing device 102 via the communication network 114, in accordance with embodiments of the invention. As will be discussed with reference to FIG. 7, the server computer 112 may include internal components 702b and external components 704b, respectively. The server 112 may also operate in a cloud computing service model, such as Software as a Service (SaaS), Platform as a Service (PaaS), or Infrastructure as a Service (IaaS). The server 112 may also be located in a cloud computing deployment model, such as a private cloud, community cloud, public cloud, or hybrid cloud.

The sensor 108 may be any device or combination of devices enabled to capture physical and/or chemical characteristics of ingredients. The sensor 108 may be any device or combination of devices enabled to communicate the captured physical and/or chemical characteristics of ingredients to the program 110, for instance via a network 114, either directly or via some number of proxies or intermediary programs or devices. Sensor 108 may be, for example, an olfactory sensor, digital scale, digital thermometer, microphone, video camera, etc. Sensor 108 may represent any number or combination of sensors. In some embodiments of the invention, a sensor 108 may comprise at least one olfactory sensor and a digital scale. In some embodiments of the invention, a sensor 108 may be used to help determine the freshness of an ingredient and scan an ingredient and/or a dish for aroma visualization.

In some embodiments of the invention, the program 110 may visualize different aromas emanating from a user's dish at each step of the recipe profile and display them, using artificial intelligence systems such as IBM Crypto Anchor Verify™ (Crypto Anchor Verifier™ and all Crypto Anchor Verified™_based trademarks and logos are trademarks or registered trademarks of International Business Machines Corporation, and/or its affiliates). The program 110 may visualize different aromas by using a sensor 108 to scan an ingredient and/or dish to capture the components and sub-components. Aroma visualization may allow a user to visually identify the make-up of an ingredient and/or a dish by graphically representing the ingredients/culinary processes that are components of the aroma.

In some embodiments of the invention, the program 110 may determine the freshness of an ingredient using artificial intelligence systems such as IBM Crypto Anchor Verify™ (Crypto Anchor Verifier™ and all Crypto Anchor Verified™_based trademarks and logos are trademarks or registered trademarks of International Business Machines Corporation, and/or its affiliates). Freshness detection may allow a user to check the aroma of each ingredient in a recipe profile to determine how fresh an ingredient is before a user begins executing a recipe profile. Freshness detection can be determined by using a sensor 108 to capture the aroma of an ingredient and then comparing the captured aroma to the ingredient's aroma profile comprised within the aroma composition database. An ingredient's aroma profile may comprise the different aromas of an ingredient at multiple stages of freshness. Freshness detection may be used to help a user better understand the qualify/freshness of an ingredient.

The database 116 may be a digital repository capable of handling data storage and data retrieval. The database can be present in the server 112 and/or the client computing device 102 and/or any other location in the network 114. The database 116 may comprise the user and/or diner profiles, recipe profiles, and/or the aroma composition database. The aroma composition database comprises aroma profiles uploaded by users and experts for each of a number of ingredients and/or dishes. The database 116 can be accessible by the social platform.

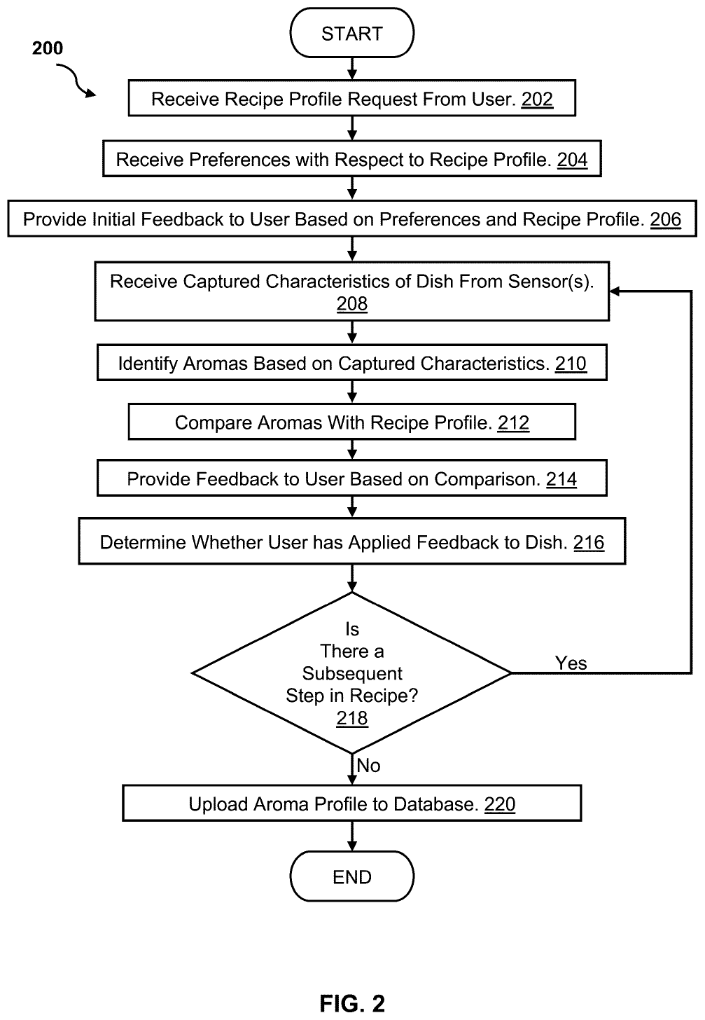

According to the present embodiment, the program 110 may be a program capable of utilizing data from one or more sensors 108, preferences and feedback to assist a user in the preparation and completion of a dish. The program 110 assists the user through capturing aromas of an ingredient/dish using a variety of sensors 108, comparing aroma profiles of a dish to aroma profiles of a recipe profile at each step of the recipe profile, receiving feedback during the preparation of a recipe profile, and suggesting modifications to the recipe profile based on the feedback. The program 110 may be located on client computing device 102 or server 112, or both, and/or on any other device located within network 114. Furthermore, the program 110 may be distributed in its operation over multiple devices, such as client computing device 102 and server 112. The social sensory recipe method is explained in further detail below with respect to FIG. 2. The program 110 may serve as a standalone application and may be platform agnostic. The program 110 may also be a portable API framework or service if another application integrates the aroma profile capturing and comparison capabilities into their existing application.

Referring now to FIG. 2, an operational flowchart illustrating a social sensory recipe process 200 is depicted according to at least one embodiment. At 202, the program 110 receives a recipe profile request from a user. A user can be any person preparing a dish corresponding to a recipe profile. A recipe profile request can comprise receiving a request from a user to make a recipe profile and/or a dish associated with the recipe profile. Receiving a recipe profile request from a user may involve the program 110 receiving a recipe profile request from the online platform in response to a user selecting a recipe profile from the online platform or a user selecting a saved or previously viewed recipe profile from the user's own profile. A recipe profile may be a set of data corresponding to a dish and the preparation of the dish. A recipe profile can represent a recipe for making a dish. A dish may herein refer to the food product being prepared by a user in accordance with the recipe profile. In addition, a recipe profile can include the aroma profile of the dish at each step and may also include aroma values associated with each ingredient for a recipe profile step. The aroma profile of a dish can comprise the aromas of a dish and one or more aroma values associated with ingredients comprising the dish representing the contribution of the ingredient to the aroma of the dish. An ingredient's aroma profile may comprise the different aromas of an ingredient at multiple stages of freshness. A recipe profile may also include other information pertaining to the dish, such as portion sizes of ingredients comprising the dish, weights of ingredients comprising the dish, cooking temperatures associated with steps of the recipe, suggestions for carrying out steps of the recipe profile, reviews of the dish by users, diners, and other individuals, etc.

At 204, the program 110 receives diner preferences with respect to the recipe profile. The program 110 may receive diner preferences from the database and/or the online platform. A diner may be a person for whom a user is preparing a dish. Diner preferences may be information the program 110 examines when a recipe profile request is received in order to determine if modifications to the recipe profile will be needed. Diner preferences may be general, such as a diner's ingredient likes/dislikes, including a diner's desire for a greater quantity of a certain ingredient in all dishes prepared for him/her, or a diner's medical conditions that are triggered by certain ingredients, such as allergies. For example, a diner's profile may include the fact that the diner is allergic to basil, and as such, list that basil needs to be excluded from all recipe profiles prepared for them. Additionally, diner preferences may be specific, such as diner feedback. Diner feedback may be comprised of suggested modifications to a recipe profile based on the diner previously consuming the dish corresponding to the recipe profile and communicating dish modifications to the program 110. Diner feedback may be added to the diner's profile. For example, a diner who had previously consumed a steak dish may communicate diner feedback to the program 110 stating that the diner would prefer the steak to be cooked well-done in the future, which may be uploaded to the diner's profile. The next time the program 110 receives a recipe profile request from a user who is preparing the dish for the diner, the program 110 may examine the database 116 and/or online platform, determine that the recipe profile needs to be modified based on checking a list of uploaded modifications comprised within the diner's profile, and rework the recipe profile to alert a user that the steak should be cooked well-done. Some diner preferences, such as medical conditions, may require a user to respond to an additional pop-up or alert, while other diner preferences, such as ingredient likes/dislikes and diner feedback, may be merely suggested recipe profile modifications that a user could accept or reject. In some embodiments, a diner may be a user and/or a user may be a diner. In some embodiments, a diner is not a user and/or a user is not a diner. In some embodiments, for example, where a user is not a diner, the program 110 may receive user preferences in addition to diner preferences with respect to the recipe profile. User preferences may record certain ingredients which risk causing an illness/condition to the user because of skin contact and/or smell, such as contact allergies and hyperosmia respectively.

At 206, the program 110 provides initial feedback to a user based on diner preferences and the recipe profile requested. Initial feedback may be feedback communicated to a user before the user begins executing a recipe profile. The initial feedback may be created based on diner preferences and the applicability of those diner preferences to the requested recipe profile. Determining the applicability of diner preferences to the requested recipe profile may require the program 110 to assess the requested recipe profile's ingredients and use the assessment in the determination of whether any of the ingredients are encompassed within the diner's preferences, to determine if the requested recipe profile needs to be adjusted to accommodate to the diner's preferences before a user begins executing the recipe profile. After determining that the diner preferences apply to the requested recipe profile, the program 110 may determine what aspects of the recipe profile require modification. For example, the program 110 may use initial feedback regarding ingredients, to make modifications to the starting ingredients, such as eliminating or adjusting a starting ingredient. The program 110 can automatically recognize whether adding/subtracting certain ingredients or new additional steps will change how a different step will be performed. The initial feedback may comprise suggestions regarding modifications to the recipe profile, such as the addition/subtraction of ingredients, changes in subsequent recipe profile steps, etc. For example, if a recipe profile has pepper in it but a diner's preferences indicate a dislike for pepper, the program 110 may take that information into account and suggest that the user use less pepper and/or more of another ingredient in the modified recipe profile. Suggestions created during the initial feedback step may guide a user to the completion of a dish by modifying the recipe profile with respect to the diner preferences. In some embodiments, for example, where a user is not a diner, suggestions created during the initial feedback step may guide a user to the completion of a dish by modifying the recipe profile based on both diner preferences and user preferences.

At 208, the program 110 receives the captured characteristics of the dish with a sensor 108. The captured characteristics may comprise physical and chemical characteristics of the ingredients in the dish, for example, the smell of an ingredient or the weight of an ingredient, and the dish itself, as combined ingredients may have properties that no individual ingredient possesses, for example, bread, and may also include optical information, temperature, etc. The capturing process may be performed once per step, multiple times per step, multiple times during the preparation of the dish, and/or continuously in real-time or near-real-time by a sensor 108. The capturing process may also be triggered at the completion of a step by a user answering a prompt. The sensor 108 may be any device or combination of devices enabled to capture physical and/or chemical characteristics of ingredients. The sensor 108 may be any device or combination of devices enabled to communicate the captured physical and/or chemical characteristics of ingredients to the program 110, for instance via a network 114, either directly or via some number of proxies or intermediary programs or devices. The sensor 108 may be, for example, an olfactory sensor, digital scale, digital thermometer, microphone, video camera, etc. The sensor 108 may, for example, capture the weight of an ingredient by comprising a scale and transferring the measured weight to the program 110. Additionally, for example in embodiments where the sensor 108 comprises an olfactory sensor, the sensor 108 may capture the aroma of an ingredient, or dish, and transfer the aroma value, or values, to the program 110. In some embodiments of the invention, the program 110 may receive captured characteristics using live video and/or augmented reality devices. For example, optical sensors 108 may be used to help capture characteristics such as portion sizes. An optical sensor 108 may capture images from a video feed and the program 110 may use machine vision techniques to identify the ingredients within the captured video footage. The use of live video may allow for a user to be viewed by others while preparing a recipe profile either in real-time or near-real-time. The aroma profile of the dish being prepared may be displayed on the video display with text identifying the percentage of each ingredient in a dish. Through the social platform, others may view the user preparing a recipe profile.

Augmented reality may enable a user to interact with an expert or another user in real-time or near-real-time using augmented reality capabilities. This may allow a user to get real-time or near-real-time feedback from another user or expert. The user preparing the dish may receive this feedback via text displayed on the augmented reality device in communication with the program 110. For example, if an expert viewing a user preparing a recipe profile notices the user incorrectly performing a step, the expert may communicate real-time or near-real-time feedback to the user.

At 210, the program 110 identifies the aromas based on the captured characteristics in step 208. The program 110 can identify aromas by comparing the captured characteristics against the aroma values comprised within the aroma composition database to identify aromas in the user's dish and/or individual components of the user's dish. The comparison may involve the program 110 matching the individual aroma values of the dish measured at the current step by the olfactory sensor to the aroma values of ingredients comprised within the aroma composition database. For example, the program 110 may take an aroma value captured by an olfactory sensor and compare it to an aroma value within the aroma composition database to identify the ingredient the aroma value represents.

At 212, the program 110 compares the identified aromas of the dish with the recipe profile. The comparison may involve a quantitative analysis comparing the aroma profile of the user's dish to the aroma profile comprised within the recipe profile. The program 110 may perform the aroma profile comparison by matching the aroma profile of the user's dish to an aroma profile comprised within the recipe profile to determine the current step the user is on in the recipe profile. Once the program 110 matches the aroma profile of the dish with the aroma profile corresponding to the current step comprised within the recipe profile, the program 110 may compare the aroma values of each ingredient in the aroma profile of the user's dish with the aroma values of each ingredient in the aroma profile at the matching step within the recipe profile. For example, the program 110 may determine, based on the captured characteristics, that the current aroma value of chili pepper in a user's dish is 32% and that the aroma value of chili pepper in a recipe profile's dish is 42%, and as such, automatically determine suggested modifications for a user to make to the recipe profile to result in the aroma value of the chili pepper in the user's dish to match the aroma value of the chili peppers in the recipe profile. Additionally, a user interface may display the current aroma value of chili pepper in a user's dish as 32% in one column and display the aroma value of chili pepper in a recipe profile's dish as 42% in another column.

At 214, the program 110 provides feedback to the user based on the comparison performed in step 212. The feedback may be generated based on the differences in the aroma profiles between the user's dish and the recipe profile. The program 110 can automatically determine suggested modifications for a user to make to the recipe profile to result in the aroma profile of the user's dish matching the aroma profile comprised within the recipe profile. The program 110 may present the suggested modifications to the user as feedback. The program 110 may provide feedback by displaying alerts/flags to the user. Alerts and flags may be pop-up prompts on the display with text identifying the feedback. For example, a feedback request may be displayed to the user as they are executing a recipe profile step that requires the addition of oregano but where no oregano is detected by the program 110 during the step, and thus, the user may be alerted to the omission of oregano and consider adding the oregano. In some embodiments, the program 110 may provide feedback by means of emitting sounds/speech through the user's mobile or computing device speakers, or by means of initiating vibration of the user's mobile or computer device. The feedback may comprise suggestions regarding modifications to the recipe profile, such as addition/subtraction of ingredients, changes in subsequent recipe profile steps, etc. For example, if the program 110 determines that the current aroma value of chili pepper in a user's dish is 32% and that the aroma value of chili pepper in a recipe profile's dish is 42%, the program 110 may suggest the addition/subtraction of ingredients to get the aroma value of chili pepper in the user's dish to match the recipe profile's aroma value of 42%.

At 216, the program 110 determines whether the user has applied the given feedback to the dish. The program 110 may determine if the user has applied the given feedback by having the user answer a prompt or based on a sensor 108 detecting changes in the dish's aroma profile. A sensor 108 may detect changes in the dish's aroma profile and may determine, for example, how long a dish has been cooked based on the aroma changes. Additionally, the program 110 may determine if recommended feedback was applied based on if a sensor 108, for example, detects an aroma not yet present in the dish, meaning a new ingredient was added to the dish. The program 110 may consider the user's answer to a prompt or the aroma profile of the dish regarding suggestions for modifying subsequent steps in the recipe profile. The program 110 may modify a recipe profile based on the addition/subtraction of certain ingredients. The program 110 may modify the recipe profile by changing/adding/removing steps. For example, if a recipe profile called for a dish to be boiled for fifteen minutes because of the presence of a certain ingredient and the program 110 determined that a user did not add the ingredient, the program 110, considering the lack of the ingredient, may modify the recipe profile so that the dish is only boiled for ten minutes.

Next, at 218, the program 110 determines if there is a subsequent step in the recipe profile. The program 110 may determine what step a user is on based on the aromas captured by the sensor 108 or by a user manually selecting a prompt to move between steps. The program 110 makes a step determination by checking the aroma profile of the user's dish and comparing it to the aroma profile comprised within the recipe profile. For example, the program 110 may determine that the aroma profile of the user's dish matches the aroma profile comprised within the recipe profile at step three and thus, determine that the user has completed step three. Subsequently, the program 110 may check the recipe profile, detect that the recipe profile consists of four steps, and therefore, the program 110 would determine that the user should proceed to step four.

According to one implementation, if the program 110 determines that there is a subsequent step in the recipe profile (step 218, “Yes” branch), the program 110 may continue to step 208 to receive the captured characteristics of the dish from a sensor 108. If the program 110 determines that there is not a subsequent step in the recipe profile (step 218, “No” branch), the program 110 may continue to step 220 to share the aroma profile of the finished dish with the online platform.

At 220, the program 110 uploads the aroma profile to the aroma compositions database comprised in the database 116. Uploading the aroma profile may allow the aroma profile to be stored and shared on the online platform. The aroma composition database comprises aroma profiles of one or more recipe profiles, which are uploaded by experts. In some embodiments, the aroma profile may be uploaded directly to the social platform and downloaded to the database 116 from the social platform. In some embodiments, the aroma profile may be uploaded to both the aroma compositions database and the social platform. The program 110 may allow for others to view the uploaded aroma profiles and may enable the sharing of aroma profiles, allowing for comparisons between users' dishes.

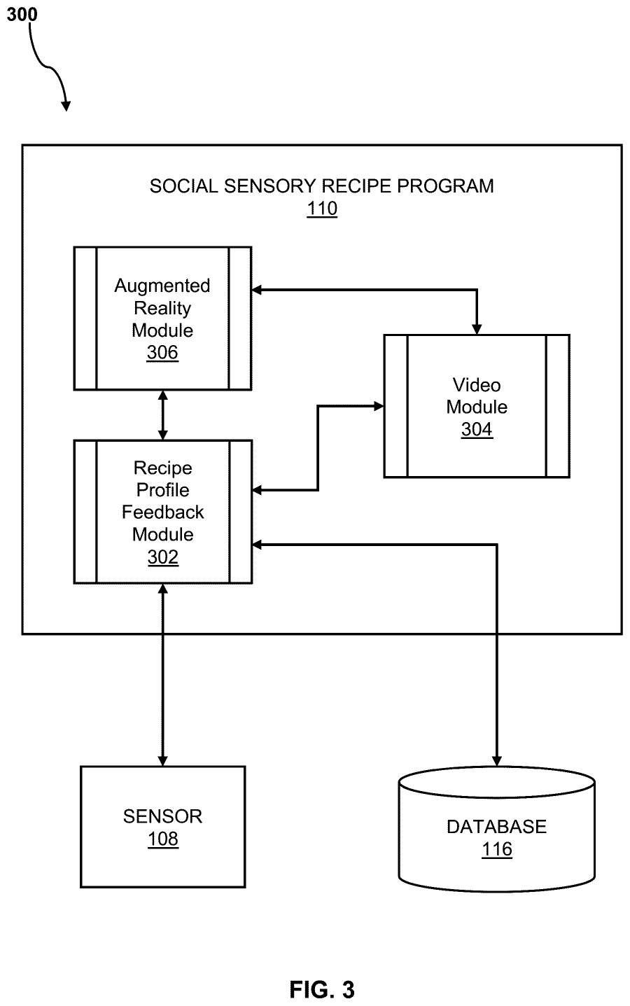

Referring now to FIG. 3, a system diagram illustrating an exemplary program environment 300 of an implementation of a social sensory recipe process 200 is depicted according to at least one embodiment. Here, the program 110 comprises a recipe profile feedback module 302, a video module 304 and an augmented reality module 306. The exemplary program environment 300 details the interactions between the recipe profile feedback module 302 and the augmented reality module 306, the recipe profile feedback module and the video module 304, and the video module 304 and the augmented reality module 306. Additionally, the exemplary program environment 300 details the interactions between recipe profile feedback module 302 and a sensor 108 via the communication network 114, and the recipe profile feedback module 302 and the database 116.

Recipe profile feedback module 302 may provide feedback based on information received from a sensor 108, database 116, video module 304, and augmented reality module 306. In some embodiments, recipe profile feedback module 302 may provide feedback based on information received from a sensor 108, or modules 304, 306, and/or 308. For example, information received from a sensor 108, such as aromas and ingredient weight, is communicated to the recipe profile feedback module 302. The recipe profile feedback module 302 may communicate feedback to a user. The recipe profile feedback module 302 may communicate with a user via text and/or graphical elements on the user's mobile device or computing device, vibrations on the user's device, sounds, or synthetic/recorded speech played from the user's device speakers. In some embodiments, recipe profile feedback module 302 may also provide initial feedback based on information received from a sensor 108, database 116, video module 304, and augmented reality module 306.

The video module 304 may allow a user to compare their dish's aroma profile to that of another user or expert, such as their mother or their friend. Through the social platform, others may view the user preparing a recipe profile. The video feature may be live or prerecorded. The use of live video may allow for a user to watch another user or expert create a dish in real-time or near-real-time. A prerecorded video may allow for a user to watch how another user or expert previously prepared a recipe profile. The aroma profile of the dish being prepared may be displayed on the video display with text identifying the percentage of each ingredient in a dish. In some embodiments of the invention, the program 110 may receive captured characteristics using live video. For example, optical sensors 108 may be used to help capture characteristics such as portion sizes. An optical sensor 108 may capture images from a video feed and the program 110 may use machine vision techniques to identify the ingredients within the captured video footage.

The augmented reality module 306 may enable a user to interact with an expert or another user in real-time or near-real-time using augmented reality capabilities. Augmented reality may be enabled by hardware in use the program 110, such as a headset with a sensor 108 incorporated in it or a mobile device with a sensor 108 incorporated in it. This may allow a user to get real-time or near-real-time feedback from another user or expert. The user preparing the dish may receive this feedback via text displayed on the augmented reality device in communication with the program 110. For example, if an expert viewing a user preparing a recipe profile notices the user incorrectly performing a step, the expert may communicate real-time or near-real-time feedback to the user.

The database 116 may be a digital repository capable of data storage and data retrieval. The database can be present in the server 112 and/or the client computing device 102 and/or any other location in the network 114. The database 116 may comprise the user and/or diner profiles, recipe profiles, and/or the aroma composition database. The aroma composition database may comprise aroma profiles uploaded by users and experts for each of a number of ingredients and/or dishes. The database 116 can be accessible by the social platform.

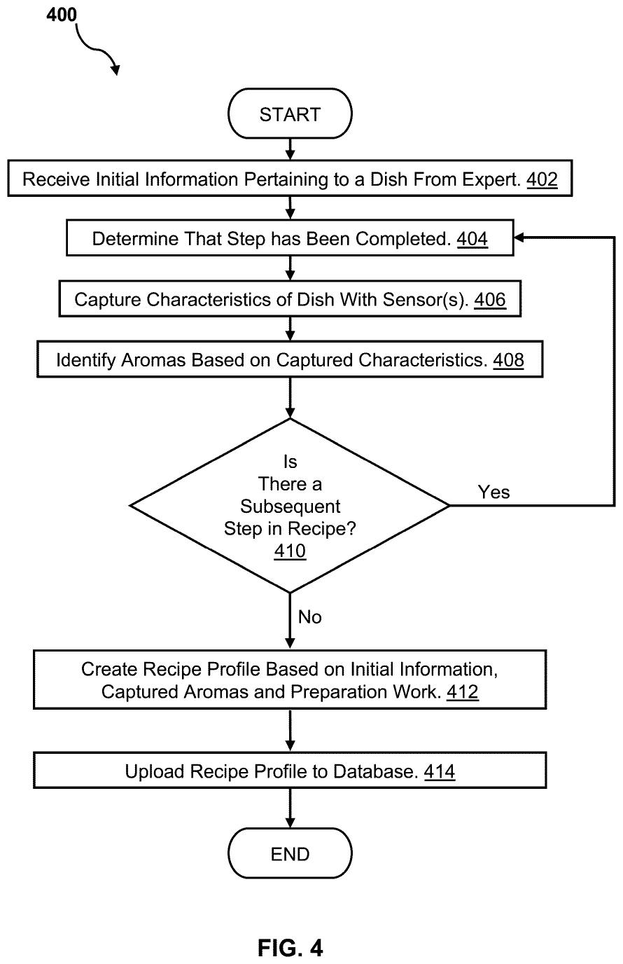

Referring now to FIG. 4, an operational flowchart illustrating a recipe profile creation process 400 is depicted according to at least one embodiment. At 402, the program 110 receives initial information pertaining to a dish from an expert. An expert can be any person who is uploading a recipe profile to the online platform. Initial information can be a set of data corresponding to the preparation of a dish entered by the expert prior to preparing the dish. Initial information may be part of a recipe profile comprising information for the making of a dish, and may correspond to the steps of the recipe profile, the kitchen appliances/tools needed for the recipe profile, what ingredients and how much of each ingredient are needed for the recipe profile, cooking temperatures and times, feedback to help users complete certain steps of the recipe profile, etc. A dish may herein refer to the food product being prepared by a user in accordance with the recipe profile.

At 404, the program 110 determines that the recipe profile step has been completed. The program 110 can determine that a recipe profile step has been completed by having an expert manually answer a completed step prompt. For example, the program 110 may receive a “Yes” response to a “Finished Step?” prompt from the expert and thus, the program 110 can determine that the expert has completed step three.

At 406, the program 110 receives the captured characteristics of the dish with a sensor 108. The captured characteristics may comprise physical and chemical characteristics of the ingredients in the dish, for example, the smell of an ingredient or the weight of an ingredient, and the dish itself, as combined ingredients may have properties that no individual ingredient possesses, for example, bread, and may also include optical information, temperature, etc. The capturing process may be performed once per step, multiple times per step, multiple times during the preparation of the dish, and/or continuously in real-time or near-real-time by a sensor 108. The capturing process may also be triggered at the completion of a step by a user answering a prompt. The sensor 108 may be any device or combination of devices enabled to capture physical and/or chemical characteristics of ingredients. The sensor 108 may be any device or combination of devices enabled to communicate the captured physical and/or chemical characteristics of ingredients to the program 110, for instance via a network 114, either directly or via some number of proxies or intermediary programs or devices. The sensor 108 may be, for example, an olfactory sensor, digital scale, digital thermometer, microphone, video camera, etc. The sensor 108 may, for example, capture the weight of an ingredient by comprising a scale and transferring the measured weight to the program 110. Additionally, for example in embodiments where the sensor 108 comprises an olfactory sensor, the sensor 108 may capture the aroma of an ingredient, or dish, and transfer the aroma value, or values, to the program 110. In some embodiments of the invention, the program 110 may receive captured characteristics using live video and/or augmented reality devices. For example, optical sensors 108 may be used to help capture characteristics such as portion sizes. An optical sensor 108 may capture images from a video feed and the program 110 may use machine vision techniques to identify the ingredients within the captured video footage. The use of live video may allow for a user to be viewed by others while preparing a recipe profile either in real-time or near-real-time. The aroma profile of the dish being prepared may be displayed on the video display with text identifying the percentage of each ingredient in a dish. Through the social platform, others may view the user preparing a recipe profile.

At 408, the program 110 identifies the aromas based on the captured characteristics in step 406. The program 110 can identify aromas by comparing the captured characteristics against the aroma values comprised within the aroma composition database to identify aromas in the user's dish and/or individual components of the user's dish. The comparison can involve the program 110 matching the individual aroma values of the dish measured at the current step by the olfactory sensor to the aroma values of ingredients comprised within the aroma composition database. For example, the program 110 may take an aroma value captured by an olfactory sensor and compare it to an aroma value within the aroma composition database to identify the ingredient the aroma value represents.

Next, at 410, the program 110 determines if there is a subsequent step in the recipe profile. The program 110 can determine what step an expert is on based on an expert manually selecting a prompt to move between steps. For example, the program 110 may receive a “Yes” response to a “Finished Step?” prompt from the expert and thus, the program 110 can determine that the expert has completed step three. Subsequently, the program 110 may check the initial information previously entered by the expert, detect that the recipe profile consists of four steps, and therefore, the program 110 may determine that the expert should proceed to step four.

According to one implementation, if the program 110 determines that there is a subsequent step in the recipe profile (step 410, “Yes” branch), the program 110 may continue to step 404 to determine if the subsequent step is completed. If the program 110 determines that there is not a subsequent step in the recipe profile (step 410, “No” branch), the program 110 may continue to step 412 to create a recipe profile based on the initial information, captured aromas, and preparation work.

At 412, the program 110 creates a recipe profile based on the initial information, captured aromas, and preparation work. The program 110 can create a recipe profile by saving the set of data corresponding to a dish and the preparation of the dish entered by the expert and captured by a sensor 108, into a single recipe profile. A recipe profile can be associated with a recipe for making a dish and corresponding to recipe steps requiring multiple ingredients. In addition, a recipe profile may include the aroma profile of the dish at each step and may also include aroma values associated with each ingredient for a recipe profile step. The aroma profile of a dish can comprise the aromas of a dish and one or more aroma values associated with ingredients comprising the dish representing the contribution of the ingredient to the aroma of the dish. An ingredient's aroma profile may comprise the different aromas of an ingredient at multiple stages of freshness. A recipe profile may also include other information pertaining to the dish, such as portion sizes of ingredients comprising the dish, weights of ingredients comprising the dish, cooking temperatures associated with steps of the recipe, suggestions for carrying out steps of the recipe profile, reviews of the dish by users, diners, and other individuals, etc. The program 110 may then format the set of data so that the recipe profile can be displayed on an interactive user interface.

At 414, the program 110 uploads the recipe profile to the database 116. Uploading the recipe profile may allow the recipe profile to be stored and shared on the online platform. The database comprises the user and diner profiles, recipe profiles, and the aroma compositions database. In some embodiments, the recipe profile may be uploaded directly to the social platform and downloaded to the database 116 from there. In some embodiments, the recipe profile may be uploaded to both the database 116 and the social platform. The program 110 may allow for others to view the uploaded recipe profiles and may enable the sharing of recipe profiles, allowing for comparisons between users' dishes.

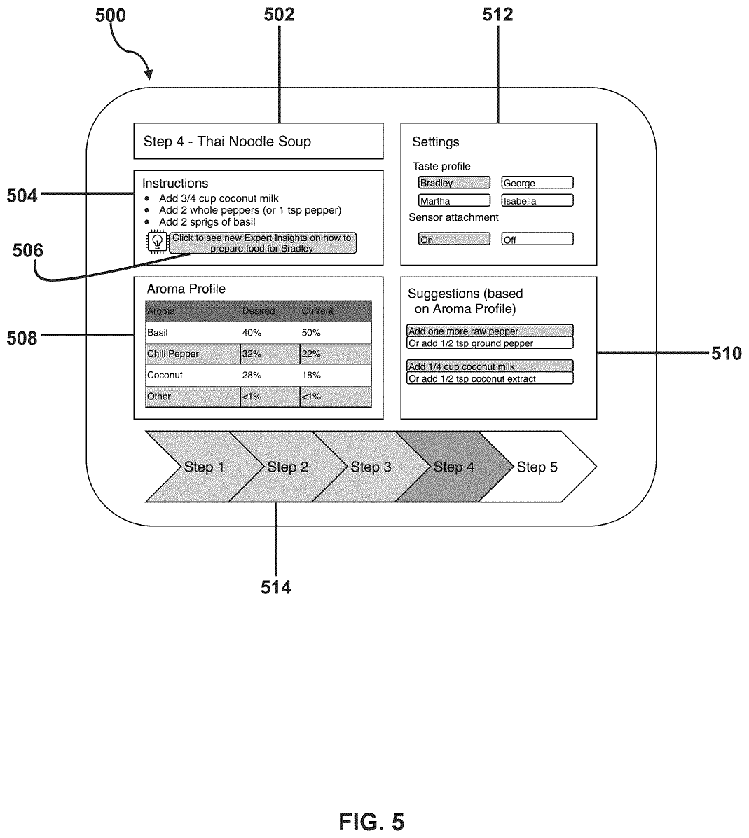

Referring now to FIG. 5, a visual representation of a graphical user interface of the system 500 is depicted according to at least one embodiment. The graphical user interface of the system 500 may display one or more trays 502, 504, 506, 508, 510, and 512. A tray is a defined region within the graphical user interface. One tray 502 may display the recipe profile title and current recipe profile step. Next, another tray 504 may display the recipe profile instructions and an expert insights tray 506. The recipe profile instructions may display any number of steps at a time. The expert insights tray 506 may display text engaging a prompt for a user to proceed to the graphical user interface of the system 600. Tray 508 may display the aroma profile of the dish at the current step and may also display the aroma profile comprised within the recipe profile at the current step as well. Tray 510 may display feedback based on the comparison of the aroma profile of the dish to the aroma profile comprised within the recipe profile at the current step. The feedback may comprise modifications that the user may make to the dish. Tray 512 may display information relating to diner preferences and sensor 108 connectivity. In tray 512, a user may add a diner or diners that the user will be preparing a dish for. The program 110 may pull multiple diners' preferences from respectively associated diner profiles and modify a recipe profile accordingly. In the case of competing diner preferences, the program 110 may notify a user using, for example, pop-up prompts on the display with text identifying the competing diner preferences. The user may choose what diner preferences to adjust or eliminate in order to resolve the competing diner preferences. The program 110 may then modify a recipe profile based on the diner preferences the user chooses to adjust or eliminate. In some embodiments, for example, where a user is not a diner, tray 512 may display user preferences in addition to diner preferences with respect to the recipe profile. Tray 514 may display a step progress bar and indicate based on shade and/or coloring which steps the user has completed, which step the user is currently on, and which steps the user has left. For example, the steps the user has completed may be lightly shaded, the step the user is currently on may be shaded darker, and the steps the user has left may not be shaded.

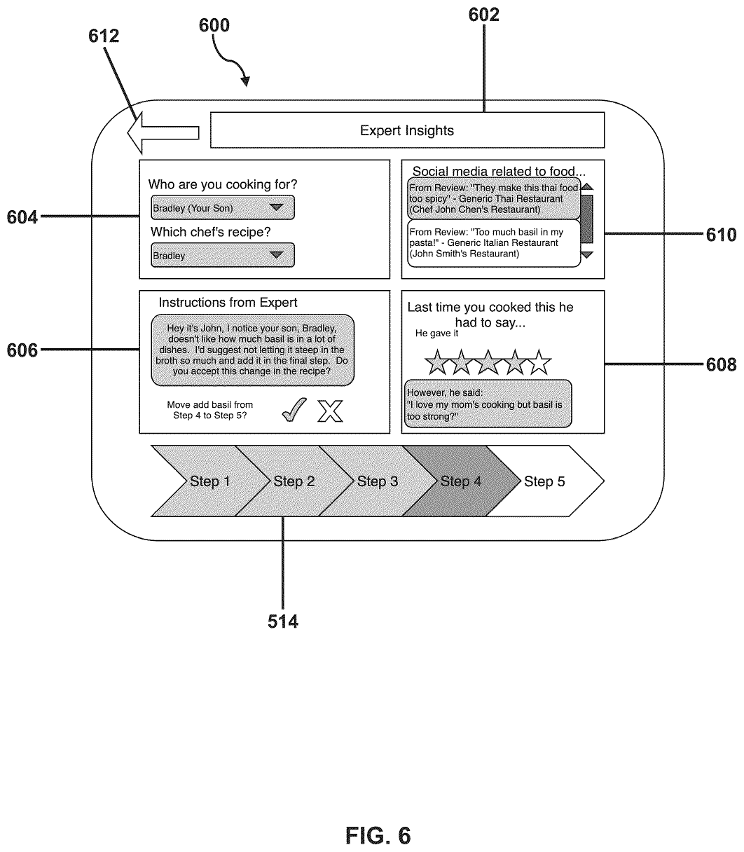

Referring now to FIG. 6, a visual representation of a graphical user interface of the system 600 is depicted according to at least one embodiment. The graphical user interface of the system 600 may display one or more trays 602, 604, 606, 608, 610, 612, and 514. A tray is a defined region within the graphical user interface. One tray 602 may display the title of the graphical user interface, for example expert insights, of the system 600. Next, another tray 604 may display text relating to a diner or diners the user is preparing a dish for and the recipe profile the user is following. Tray 606 may display suggested modifications from an expert. The suggested modifications may comprise modifications to the recipe profile that are obtained directly from an expert and are specifically related to the recipe profile and the diner preferences. Tray 606 may also display a prompt in which the user may choose to accept or reject the suggested modifications from the expert. Tray 608 may display feedback from an individual who has previously consumed the dish prepared from the recipe profile. The feedback may comprise text and/or a rating based on, for example, a 5-star rating system. Tray 610 may display reviews from the social media platform. Reviews from the social media platform are obtained by the program 110 pulling reviews from integrated outside platforms and may contain text relating to general reviews of the dish being prepared in the recipe profile and/or pictures. For example, the general reviews may relate to feedback on how a dish is prepared at a restaurant. Tray 612 may display a prompt, such as an arrow, engaging a user action to return to the graphical user interface of the system 500. Tray 514 may display a step progress bar and indicate based on shade and/or coloring which steps the user has completed, which step the user is currently on, and which steps the user has left. For example, the steps the user has completed may be lightly shaded, the step the user is currently on may be shaded darker, and the steps the user has left may not be shaded.

It may be appreciated that FIGS. 2-6 provide only illustrations of individual implementations and do not imply any limitations with regard to how different embodiments may be implemented. Many modifications to the depicted environments may be made based on design and implementation requirements.

FIG. 7 is a block diagram 700 of internal and external components of the client computing device 102 and the server 112 depicted in FIG. 1 in accordance with an embodiment of the present invention. It should be appreciated that FIG. 7 provides only an illustration of one implementation and does not imply any limitations with regard to the environments in which different embodiments may be implemented. Many modifications to the depicted environments may be made based on design and implementation requirements.

The data processing system 702, 704 is representative of any electronic device capable of executing machine-readable program instructions. The data processing system 702, 704 may be representative of a smart phone, a computer system, PDA, or other electronic devices. Examples of computing systems, environments, and/or configurations that may represented by the data processing system 702, 704 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

The client computing device 102 and the server 112 may include respective sets of internal components 702 a, b and external components 704 a, b illustrated in FIG. 7. Each of the sets of internal components 702 include one or more processors 720, one or more computer-readable RAMs 722, and one or more computer-readable ROMs 724 on one or more buses 726, and one or more operating systems 728 and one or more computer-readable tangible storage devices 730. The one or more operating systems 728, the program 110 in the client computing device 102, and the program 110 in the server 112 are stored on one or more of the respective computer-readable tangible storage devices 730 for execution by one or more of the respective processors 720 via one or more of the respective RAMs 722 (which typically include cache memory). In the embodiment illustrated in FIG. 7, each of the computer-readable tangible storage devices 730 is a magnetic disk storage device of an internal hard drive. Alternatively, each of the computer-readable tangible storage devices 730 is a semiconductor storage device such as ROM 724, EPROM, flash memory or any other computer-readable tangible storage device that can store a computer program and digital information.

Each set of internal components 702 a, b also includes a R/W drive or interface 732 to read from and write to one or more portable computer-readable tangible storage devices 738 such as a CD-ROM, DVD, memory stick, magnetic tape, magnetic disk, optical disk or semiconductor storage device. A software program, such as the program 110, can be stored on one or more of the respective portable computer-readable tangible storage devices 738, read via the respective R/W drive or interface 732, and loaded into the respective hard drive 730.

Each set of internal components 702 a, b also includes network adapters or interfaces 736 such as a TCP/IP adapter cards, wireless Wi-Fi interface cards, or 3G or 4G wireless interface cards or other wired or wireless communication links. The program 110 in the client computing device 102 and the program 110 in the server 112 can be downloaded to the client computing device 102 and the server 112 from an external computer via a network (for example, the Internet, a local area network or other, wide area network) and respective network adapters or interfaces 736. From the network adapters or interfaces 736, the program 110 in the client computing device 102 and the program 110 in the server 112 are loaded into the respective hard drive 730. The network may comprise copper wires, optical fibers, wireless transmission, routers, firewalls, switches, gateway computers and/or edge servers.

Each of the sets of external components 704 a, b can include a computer display monitor 744, a keyboard 742, and a computer mouse 734. External components 704 a, b can also include touch screens, virtual keyboards, touch pads, pointing devices, and other human interface devices. Each of the sets of internal components 702 a, b also includes device drivers 740 to interface to computer display monitor 744, keyboard 742, and computer mouse 734. The device drivers 740, R/W drive or interface 732, and network adapter or interface 736 comprise hardware and software (stored in storage device 730 and/or ROM 724).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

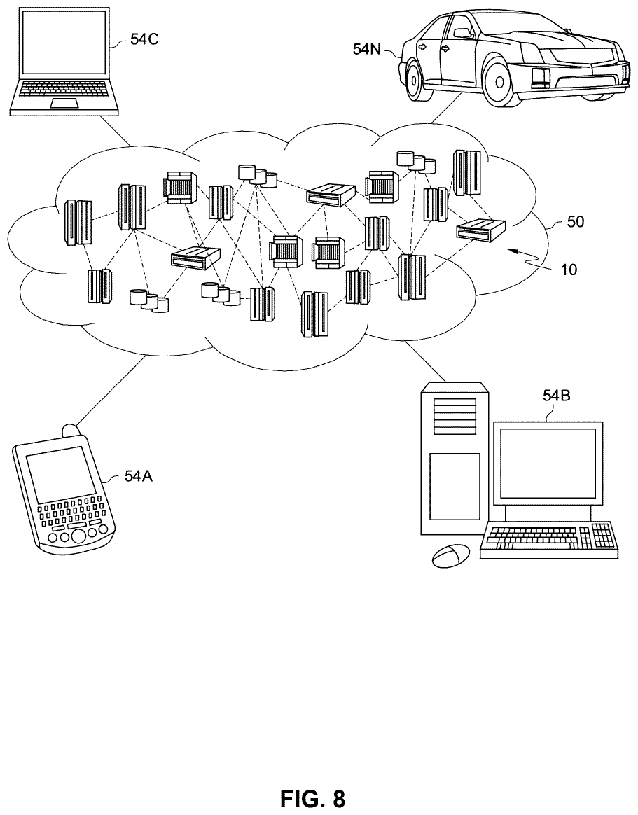

Referring now to FIG. 8, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 8 are intended to be illustrative only and that computing nodes 10 and cloud computing environment 50 can communicate with any type of computerized device over any type of network and/or network addressable connection (e.g., using a web browser).

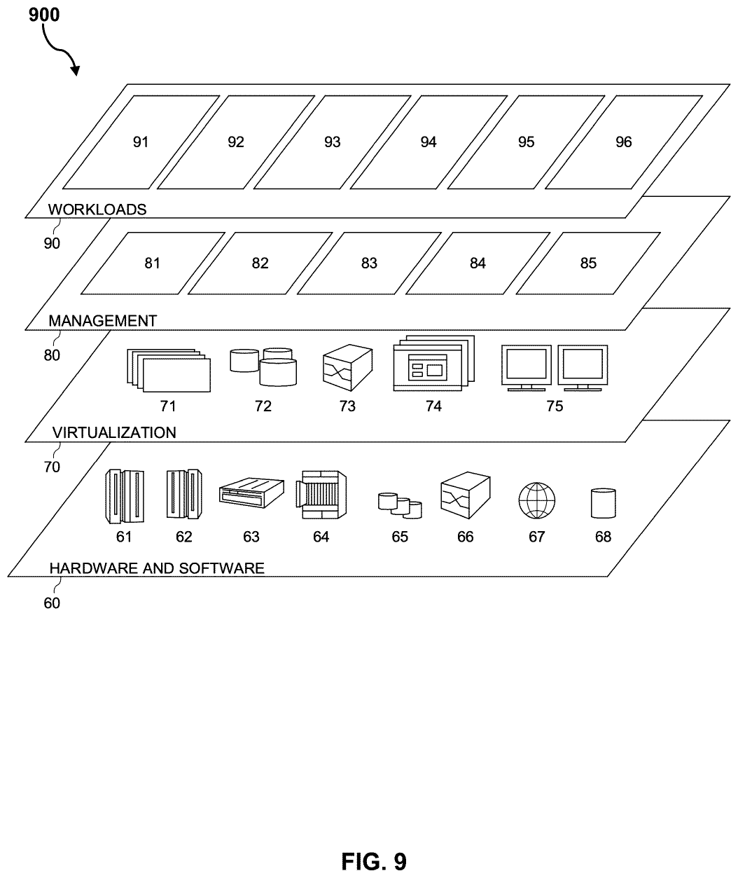

Referring now to FIG. 9, a set of functional abstraction layers 900 provided by cloud computing environment 50 is shown. It should be understood in advance that the components, layers, and functions shown in FIG. 9 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and the social sensory recipe program 96. The social sensory recipe program 96 may relate to a system, method, and/or computer program product for utilizing data from one or more sensors and feedback to assist a user in the preparation and completion of a dish. The program 96 assists the user through capturing aromas of an ingredient/dish using a variety of sensors, comparing aroma profiles of a dish to aroma profiles of a recipe profile at each step of the recipe profile, receiving feedback during the preparation of a recipe profile, and suggesting modifications to the recipe profile based on the feedback.

The descriptions of the various embodiments of the present invention have been presented for purposes of illustration but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A processor-implemented method comprising:
capturing one or more characteristics comprising an aroma of a dish associated with a recipe using one or more sensors, wherein the one or more sensors comprise an olfactory sensor;
identifying, at two or more steps of the recipe comprising a recipe profile, one or more aroma profiles of the dish based on the captured one or more characteristics comprising the aroma;
comparing the one or more aroma profiles of the dish to one or more aroma profiles comprising the recipe profile; and
responsive to the comparing, providing feedback of the dish to a user.

2. The method of claim 1, further comprising:
responsive to receiving a recipe profile request, modifying the recipe profile based on diner preferences.

3. The method of claim 2, wherein modifying the recipe profile is further based on user preferences.

4. The method of claim 1, further comprising:
comparing the one or more aroma profiles of a user's dish to that of a different user's or an expert's dish through use of a video feature.

5. The method of claim 1, further comprising:
receiving, during execution of the recipe, real-time or near-real-time feedback from an expert or another user using augmented reality capabilities.

6. The method of claim 1, further comprising:
detecting, before or during execution of the recipe, freshness of one or more ingredients based on the captured characteristics of the dish using artificial intelligence systems.

7. The method of claim 1, further comprising:
determining whether the user has applied the feedback to the dish.

8. A computer system comprising:
one or more processors, one or more computer-readable memories, one or more computer-readable tangible storage medium, and program instructions stored on at least one of the one or more tangible storage medium for execution by at least one of the one or more processors via at least one of the one or more memories, wherein the computer system is capable of performing a method comprising:
capturing one or more characteristics comprising an aroma of a dish associated with a recipe using one or more sensors, wherein the one or more sensors comprise an olfactory sensor;
identifying, at two or more steps of the recipe comprising a recipe profile, one or more aroma profiles of the dish based on the captured one or more characteristics comprising the aroma;
comparing the one or more aroma profiles of the dish to one or more aroma profiles comprising the recipe profile; and
responsive to the comparing, providing feedback of the dish to a user.

9. The computer system of claim 8, further comprising:
responsive to receiving a recipe profile request, modifying the recipe profile based on diner preferences.

10. The computer system of claim 9, wherein modifying the recipe profile is further based on user preferences.

11. The computer system of claim 8, further comprising:
comparing the one or more aroma profiles of a user's dish to that of a different user's or an expert's dish through use of a video feature.

12. The computer system of claim 8, further comprising:
receiving real-time or near-real-time feedback from an expert or from another user using augmented reality capabilities.

13. The computer system of claim 8, further comprising:
detecting, before or during execution of the recipe, freshness of one or more ingredients based on the captured characteristics of the dish using artificial intelligence systems.

14. The computer system of claim 8, further comprising:
determining whether the user has applied the feedback to the dish.

15. A computer program product comprising:
one or more computer-readable tangible storage medium and program instructions stored on at least one of the one or more tangible storage medium, the program instructions executable by a processor to cause the processor to perform a method comprising:
capturing one or more characteristics comprising an aroma of a dish associated with a recipe using one or more sensors, wherein the one or more sensors comprise an olfactory sensor;
identifying, at two or more steps of the recipe comprising a recipe profile, one or more aroma profiles of the dish based on the captured one or more characteristics comprising the aroma;
comparing the one or more aroma profiles of the dish to one or more aroma profiles comprising the recipe profile; and
responsive to the comparing, providing feedback of the dish to a user.

16. The computer program product of claim 15, further comprising:
responsive to receiving a recipe profile request, modifying the recipe profile based on diner preferences.

17. The computer program product of claim 16, wherein modifying the recipe profile is further based on user preferences.

18. The computer program product of claim 15, further comprising:
comparing the one or more aroma profiles of a user's dish to that of a different user's or an expert's dish through use of a video feature.

19. The computer program product of claim 15, further comprising:
receiving real-time or near-real-time feedback from an expert or from another user using augmented reality capabilities.

20. The computer program product of claim 15, further comprising:
detecting, before or during execution of the recipe, freshness of one or more ingredients based on the captured characteristics of the dish using artificial intelligence systems.
